SEQUENCE LISTING (2 sequences)

SEQ ID NO: 1 - protein, 42 aa, Homo sapiens [sequence omitted]

SEQ ID NO: 2 - protein, 134 aa, Apis mellifera
IIYPGTLWCGHGNKSSGPNELGRFKHTDACCRTHDMCPDVMSAGESKHGLTNTASHTRLSCDCDDKFYDCLKNSADTISSYFVGKMYFNLIDTKCYKLEHPVTGCGERTEGRCLHYTVDKSKPKVYQWFDLRKY

(19) United States
(12) Patent Application Publication
(10) Pub. No.: US 2020/0030376 A1
(43) Pub. Date: Jan. 30, 2020

(54) CELL THERAPY COMPOSITION FOR PREVENTING OR TREATING NEURODEGENERATIVE DISEASE COMPRISING REGULATORY T CELLS AS ACTIVE INGREDIENT

(71) Applicant: GMP CO., LTD., Paju-si (KR)

(72) Inventors: Hyun-Su Bae, Seoul (KR); Hyun-Jung Baek, Seoul (KR); Min-Sook Ye, Namyangju-si (KR); Chan-Ju Lee, Goyang-si (KR)

(73) Assignee: GMP CO., LTD., Paju-si (KR)

(21) Appl. No.: 16/047,994

(22) Filed: Jul. 27, 2018

(51) Int. Cl. A61K 35/17; C12N 5/0783; A61P 25/28; A61K 38/17; A61K 38/46; C07K 14/47; C07K 14/435

(52) U.S. Cl. CPC A61K 35/17; C12N 5/0637; A61P 25/28; C07K 14/43572; A61K 38/465; C07K 14/4711; A61K 38/1716

(57) ABSTRACT

The present invention relates to a cell therapy method for treating neurodegenerative diseases, which includes administering a therapeutically effective amount of a regulatory T cell to an individual in need thereof. The regulatory T cell may be an amyloid-beta specific regulatory T cell induced by administering amyloid-beta peptide and bee venom phospholipase A2 (bvPLA2).

### SEQUENCE LISTING

This application contains a Sequence Listing which is named Sequence Listing.txt.

### BACKGROUND OF THE INVENTION

### Technical Field of the Invention

The present invention relates to a cell therapy method for treating neurodegenerative diseases, which includes administering a therapeutically effective amount of a regulatory T cell.

### Description of the Related Art

Alzheimer's disease is a kind of neurodegenerative diseases that causes the elder's dementia, and its primary symptom is a progressive loss of memory and cognition. The precise causes and mechanisms of Alzheimer's disease were not revealed, but senile plaques and neurofibrillary tangles were found in the brain of the patients. Further, studies have been conducted to reveal the death of nerve cells, the loss of synapses, and degeneration of Tau protein caused by toxic proteins such as amyloid-beta and anti-amyloid precursor protein (APP-C) and the formation mechanism of nerve fiber masses caused by hyperphosphorylation. Other studies have been conducted to establish ideal disease models and to develop new preventive and therapeutic methods. Recently, it is revealed that the neuroinflammation is prominent in Alzheimer's disease. Thus, the control of neuroinflammation for prevention and treatment of brain diseases is recognized as important fields.

Currently, there are medications that alleviate the mental and behavioral disorders induced by Alzheimer's disease, but there are no effective treatments or therapeutic agents to prevent or fundamentally treat the disease. During the last 30 years, researchers have actively conducted on the pathogenesis of Alzheimer's disease. Various therapeutic mechanisms have been studied, and a number of clinical trials have been conducted. In particular, it has been confirmed in animal models that neural cell regeneration techniques by stem cells elicit therapeutic effects against neurodegenerative diseases such as Alzheimer's disease, Parkinson's disease, and Huntington's disease. Among them, research on adult stem cells has attracted much attention since there are no immunological rejection, tumor formation, and ethical and moral problems.

Studies have been actively conducted on Alzheimer's disease (senile dementia) which is called “21st-century disease” and is the biggest issue in our aging society of the 21st century. Thus, it is predicted that its vaccine and stem cell treatment will be developed based on its causes in the short term among the brain diseases to conquer the disease to some extent. Due to the increasing aging population, brain diseases such as dementia and stroke are increasing explosively in elderly people. The development of therapeutic drugs to treat the diseases is a core technology for promoting the public health in the aging society. Further, the achievements in neuroscience research, particularly brain disease research, serve as a driving force for the national survival in the age of limitless competition, further Korea's science and technology development in achieving national goals of entry into advanced countries.

The annual market size of neurodegenerative diseases such as stroke, dementia, and Parkinson's disease, which are senile diseases, reaches 200 trillion won (Korean won) in the United States and 10 trillion won in Korea. In 2026, Korea will enter the superaged society, and thus its market size is expected to increase more than three times. In addition to the conventional compound-based therapeutic agents, the market is expanding for alternative therapeutic agents such as protein/vaccine and cell therapy in the pharmaceutical market related to the neurodegenerative disease therapeutic agent.

Therefore, the present inventors have isolated regulatory T cells (Tregs) that play a critical role in maintaining immune tolerance and immune homeostasis, thereby developing cell therapeutic agents of treating Alzheimer's disease, which are cell-transplanted into animal models with Alzheimer's disease. Moreover, the present inventors have administrated amyloid-beta peptide and bvPLA2 to animal models with Alzheimer's disease to produce amyloid-beta specific Treg cells, which were then transplanted into the animal models with Alzheimer's disease, thereby developing cell therapeutic agents of treating Alzheimer's disease. Thus, the present inventors have completed the present invention through this series of processes.

### BRIEF DESCRIPTION OF THE DRAWINGS

FIGS. 1A-1D show the cognitive ability of each mouse model, which is obtained through a Morris water maze experiment: FIG. 1A shows the results of measuring the “latency time” required for each mouse model to find a hidden platform during a four-day training session; FIG. 1B shows the results of measuring the “retention time” of each mouse model in the platform position; FIG. 1C shows the results of measuring the “time in quadrant” in which each mouse model stayed in the quadrant where the platform was positioned; and FIG. 1D shows the results of measuring the “number of crossing” that each mouse model passed the location of the platform (WT: normal mouse group; 3xTg: Alzheimer's model mouse group; 3xTg/Teff: Alzheimer's model mouse group transplanted with Teff cells; and 3xTg/Treg: Alzheimer's model mouse group transplanted with Treg cells).

FIG. 2 shows the distribution of Treg cells (Foxp3+CD4+) in the spleen of each mouse model (WT: normal mouse group; 3xTg: Alzheimer's model mouse group; 3xTg/Teff: Alzheimer's model mouse group transplanted with Teff cells; and 3xTg/Treg: Alzheimer's model mouse group transplanted with Treg cells).

FIGS. 3A-3G show the expression level of an inflammation-related cytokine in each mouse model: FIG. 3A represents IL-2, FIG. 3B represents IL-6, FIG. 3C represents IFN-γ, FIG. 3D represents IL-17A, FIG. 3E represents IL-10, FIG. 3F represents IL-4, and FIG. 3G represents TNF-α (WT: normal mouse group; 3xTg: Alzheimer's model mouse group; 3xTg/Teff: Alzheimer's model mouse group transplanted with Teff cells; and 3xTg/Treg: Alzheimer's model mouse group transplanted with Treg cells).

FIG. 4 shows amyloid-beta plaques in the brain hippocampal region of each mouse model (WT: normal mouse group; 3xTg: Alzheimer's model mouse group; 3xTg/Teff: Alzheimer's model mouse group transplanted with Teff cells; and 3xTg/Treg: Alzheimer's model mouse group transplanted with Treg cells).

FIG. 5 shows amyloid-beta plaques and microglia in the brain hippocampal region of each mouse model (WT: normal mouse group; 3xTg: Alzheimer's model mouse group; 3xTg/Teff: Alzheimer's model mouse group transplanted with Teff cells; and 3xTg/Treg: Alzheimer's model mouse group transplanted with Treg cells).

FIG. 6 shows the experimental schedule of transplanting amyloid-beta specific Treg cells.

FIGS. 7A-7D show the cognitive ability of each mouse model, which is obtained through a Morris water maze experiment: FIG. 7A shows the results of measuring the “latency time” required for each mouse model to find a hidden platform during a four-day training session; FIG. 7B shows the results of measuring the “retention” time of each mouse model in the platform position; FIG. 7C shows the results of measuring the “time in quadrant” in which each mouse model stayed in the quadrant where the platform was positioned; and FIG. 7D shows the results of measuring the “number of crossing” that each mouse model passed the location of the platform (WT: normal mouse group; 3xTg: Alzheimer's model mouse group; 3xTg/Treg-PBS: Alzheimer's model mouse group transplanted with PBS-treated Treg cells as a negative control; 3xTg/Treg-Ab: Alzheimer's model mouse group transplanted with Treg cells after Aβ vaccination; 3xTg/Treg-Ab+PLA2: Alzheimer's model mouse group transplanted with Treg cells after Aβ vaccination and PLA2 treatment; 3xTg/Treg-PLA2: Alzheimer's model mouse group transplanted with Treg cells after PLA2 treatment; and 3xTg/Treg-KLH: Alzheimer's model mouse group transplanted with Treg cells after KLH vaccination as a negative control).

FIGS. 8A-8C show the distribution of helper T cells in each mouse model through FACS analysis: FIG. 8A shows the expression level of IFN-γ+ in CD4+ T cell group; FIG. 8B shows the expression level of IL-4+ in CD4+ T cell group; and FIG. 8C shows the expression level of IL-17A+ in CD4+ T cell group (WT: normal mouse group; 3xTg: Alzheimer's model mouse group; 3xTg/Treg-PBS: Alzheimer's model mouse group transplanted with PBS-treated Treg cells as a negative control; 3xTg/Treg-Ab: Alzheimer's model mouse group transplanted with Treg cells after Aβ vaccination; 3xTg/Treg-Ab+PLA2: Alzheimer's model mouse group transplanted with Treg cells after Aβ vaccination and PLA2 treatment; 3xTg/Treg-PLA2: Alzheimer's model mouse group transplanted with Treg cells after PLA2 treatment; and 3xTg/Treg-KLH: Alzheimer's model mouse group transplanted with Treg cells after KLH vaccination as a negative control).

FIGS. 9A-9G show the expression level of an inflammation-related cytokine in each mouse model: FIG. 9A represents IL-2, FIG. 9B represents IL-6, FIG. 9C represents IFN-γ, FIG. 9D represents IL-17A, FIG. 9E represents IL-10, FIG. 9F represents IL-4, and FIG. 9G represents TNF-α (WT: normal mouse group; 3xTg: Alzheimer's model mouse group; 3xTg/Treg-PBS: Alzheimer's model mouse group transplanted with PBS-treated Treg cells as a negative control; 3xTg/Treg-Ab: Alzheimer's model mouse group transplanted with Treg cells after Aβ vaccination; 3xTg/Treg-Ab+PLA2: Alzheimer's model mouse group transplanted with Treg cells after Aβ vaccination and PLA2 treatment; 3xTg/Treg-PLA2: Alzheimer's model mouse group transplanted with Treg cells after PLA2 treatment; and 3xTg/Treg-KLH: Alzheimer's model mouse group transplanted with Treg cells after KLH vaccination as a negative control).

FIG. 10 shows amyloid-beta plaques in the brain hippocampal region of each mouse model (WT: normal mouse group; 3xTg: Alzheimer's model mouse group; 3xTg/Treg-PBS: Alzheimer's model mouse group transplanted with PBS-treated Treg cells as a negative control; 3xTg/Treg-Ab: Alzheimer's model mouse group transplanted with Treg cells after Aβ vaccination; 3xTg/Treg-Ab+PLA2: Alzheimer's model mouse group transplanted with Treg cells after Aβ vaccination and PLA2 treatment; 3xTg/Treg-PLA2: Alzheimer's model mouse group transplanted with Treg cells after PLA2 treatment; and 3xTg/Treg-KLH: Alzheimer's model mouse group transplanted with Treg cells after KLH vaccination as a negative control).

### DETAILED DESCRIPTION OF THE INVENTION

It is an aspect of the present invention to provide a cell therapy method for treating neurodegenerative diseases, which includes administering a therapeutically effective amount of a regulatory T cell to an individual in need thereof.

As used herein, the term “regulatory T cell” refers to a kind of T cells, which has the characteristics that control inflammatory responses of abnormally activated immune cells. It is also referred to as Treg. The regulatory T cell may be roughly classified into natural Treg and adaptive Treg. A CD+ CD25+ T cell, which is a natural Treg, is endowed with immunosuppressive function since when this cell is newly produced from the thymus gland and constitutes 5 to 10% of peripheral CD4+ T lymphocyte in a normal individual. Although the immunosuppressive mechanism of the natural Treg has not been clarified so far, a factor of controlling gene expression called Foxp3 have been recently found to play a critical role in the differentiation and activation of the natural Treg. Moreover, the peripheral natural T cell can be differentiated into cells which exert an immunosuppressive effect when stimulated by autoantigen or external antigen under a specific environment. This cell is called adaptive Treg or inducible Treg. The adaptive Treg includes Tr 1 secreting IL-10, Th3 secreting TGF-β, CD8 Ts, and the like.

As used herein, the term “cell therapy products” refers to a drug used for the purpose of treatment, diagnosis and prevention through a series of behaviors of in vitro multiplying and sorting living autologous, allogenic and xenogenic cells or changing the biological characteristics of cells by other means for the purpose of recovering the functions of cells and tissues. The cell therapy products has been considered a drug since 1993 in the U.S. and since 2002 in South Korea. Cell therapy products may be roughly classified into two categories: a “stem cell therapy products” for tissue regeneration or organ function recovery; and an “immune cell therapy products” for the regulation of immune response such as the suppression of immune response or hyper-immune response in vivo. In the present invention, the regulatory T cell refers to cell therapy products.

According to an embodiments of the present invention, the therapeutically effective amount of the regulatory T cell may be included in a range of 1×10⁴ cells/kg to 1×10⁸ cells/kg of body weight, preferably 1×10⁵ cells/kg to 1×10⁷ cells/kg of body weight. Cell therapy products may be administered once to several times a day, for example, to an adult.

As used herein, the term “therapeutically effective amount” refers to an amount of cell therapy products that will elicit the biological or medical response of a tissue system, animal or human that is being sought by a researcher, veterinarian, medical doctor or clinician, and encompasses an amount of cell therapy products which will relieve the symptoms of the disease or disorder being treated. It will be apparent to those skilled in the art that the amount of the cell therapy products included in the method of the present invention may vary depending upon desired therapeutic effects. Therefore, an optimal dose of the cell therapy products can be readily determined by those skilled in the art. It can be determined by taking into consideration of various factors such as kinds of disease, severity of disease, contents of other components, kinds of formulations, age, weight, health status, sex and dietary habits of patients, administration times and routes, release rates, treatment duration, and co-administered drugs. It is important to consider all of these factors to include the amount that can achieve the maximum effect in a minimal amount without side effects.

As used herein, the term “individual” refers to any mammalian species that needs treatment, observation or experiment, preferably human.

According to an embodiments of the present invention, the regulatory T cell is administered intravenously, intraperitoneally, intramuscularly, subcutaneously or intradermally. The administration route of the cell therapy products may be any conventional route as long as the cell therapy products may reach the target tissue. It may, but be not limited to, be parenteral administration. Further, the cell therapy products may be administered by an arbitrary device capable of moving the cell therapy products to target cells.

According to an embodiment of the present, the neurodegenerative diseases may be selected from the group consisting of Alzheimer's disease, Huntington's disease, and Parkinson's disease.

As used herein, the term “neurodegenerative diseases” includes apoplexy, a stroke, dementia, Alzheimer's disease, Parkinson's disease, Huntington's disease, Pick's disease or Creutzfeld-Jakob's disease.

According to an embodiment of the present, the regulatory T cell may be an amyloid-beta specific regulatory T cell induced by administering amyloid-beta peptide and bee venom phospholipase A2 (bvPLA2).

According to an embodiment of the present, the amyloid-beta peptide may consist of an amino acid sequence represented by SEQ ID NO: 1.

As used herein, the term “amyloid-beta” refers to a primary component of the amyloid-beta plaque found in the brain of a patient with Alzheimer's disease. It is known that the amyloid-beta is a peptide comprising 36 to 43 amino acids and is made of an amyloid precursor protein (APP). The amyloid precursor proteins can be degraded by β-secretase and γ-secretase.

According to an embodiment of the present, the bee venom phospholipase A2 (bvPLA2) may consist of an amino acid sequence represented by SEQ ID NO: 2.

As used herein, the term “bee venom phospholipase A2 (bvPLA2)”, which means PLA2, refers to one of the components of the bee venom mixture. PLA2 refers to an enzyme which produces fatty acids by hydrolyzation at the second carbon position of glycerol. It recognizes specifically the sn-2 acyl bond of phospholipids to catalyze the hydrolytic activity, thereby releasing arachidonic acid and lysophospholipid. PLA2 is commonly found in tissues of mammals as well as bacteria, insects and snake venom. The bvPLA2 of the present invention may, but be not limited to, be derived from bees (Apis mellifera).

### EXAMPLES

Hereinafter, the present invention will be described in more detail with reference to Examples. It will be to be understood, however, that these Examples are only for illustrative purposes and are not to be construed as limiting the scope of the present invention.

### Example 1

### 1.1. Cell Isolation and Cell Culture.

The present invention used regulatory T cells which were isolated from the pancreas of C57BL/6n mice. The mouse pancreas was removed. The removed mouse pancreas was placed in phosphate buffered saline (PBS) and ground. Then, the red blood cells thereof were removed by RBC lysis buffer. The separated splenocytes were separated into regulatory T cells (CD4+CD25+ cell group) and effector T cells (CD4+CD25− cell group) using a MACS CE4CD25 regulatory T cell isolation kit.

### 1.2. Animal Model.

3xTg-AD mice are used as an Alzheimer's disease animal model. The mouse was purchased from the Jackson laboratory (USA). The mouse has three genes associated with Alzheimer's disease including the Swedish mutant amyloid-beta precursor (APPSwe KM670/671NL), the mutant presenilin 1 (PS1 M146V), and the mutant tau (tau P301L) at the same time.

### 1.3. Method of Cell Transplantation.

In order to transplant regulatory T cells and effector T cells, the cells were injected intravenously into the tail vein of 3xTg-AD mice, an Alzheimer's disease animal model.

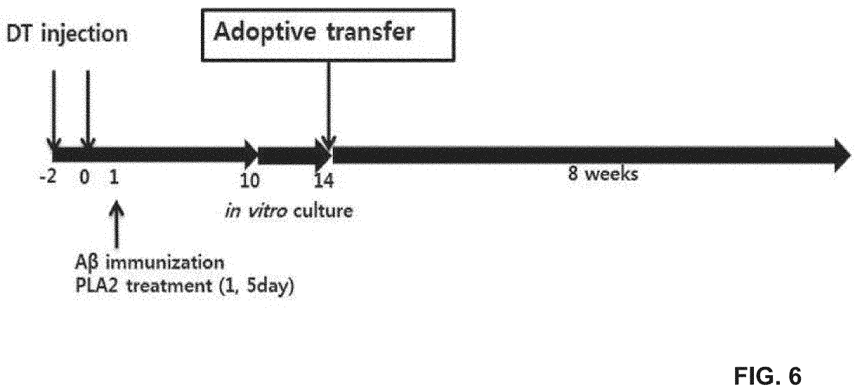

In order to transplant amyloid-beta specific Treg cells, Foxp3-DTR mouse was injected 2 days before the induction of the immune response in which the mouse was transformed in which diphtheria toxin receptor (DTR) was followed by Foxp3 gene, and Foxp3 was removed when diphtheria toxin (DT) was injected thereto. DT was injected to remove Treg cells. Then, Aβ, Aβ+PLA2, PLA2 or KLH (Keyhole Limpet Hemocyanin, control group) was treated to induce the production of Aβ-reactive Treg cells. PLA2 was injected twice on days 1 and 5. After 10 days of immune response induction, the pancreas of the mouse was treated with each of Aβ, Aβ+PLA2, PLA2 or KLH. They were cultured in vitro for 4 days. After 14 days, the cells were adoptively transferred into the vein of 3xTg-AD mice, an Alzheimer's disease animal model (See FIG. 6).

### 1.4. Morris Water Maze Experiment.

Morris water maze experiment was carried out to confirm memory and cognitive ability of each experimental group. The Morris water maze experiment was conducted by filling the cylinder with water, dividing it into quadrants, installing a hidden platform in one of the quadrants, and training mouse to find the hidden platform from the other quadrant where the hidden platform was not located for the first 4 days. On day 5, the hidden platform was removed. Then, the retention time when the mouse had stayed in the platform; the time in quadrant when the mouse had stayed in the quadrant where the platform was located; and the number of crossing that the mouse had passed the location where the platform was installed; were measured.

### 1.5. FACS Analysis.

The spleen and lymph nodes were isolated from each mouse model and analyzed by fluorescence-activated cell sorting (FACS) so as to confirm the distribution of Treg cells. The spleen and lymph node were isolated from mouse and ground. Then, the cell surface markers were stained with anti-CD4-FITC and anti-CD25-PE antibodies. The cell membranes were pierced with Fix/Perm buffer, and then the cells were stained with anti-Foxp3-PE-cy5 antibodies to confirm the distribution of Treg cells.

In order to confirm the distribution of helper T cells, the splenocytes of each mouse model were isolated and treated with ionomycin, PMA (phorbol 12-myristate 13-acetate) and golgi-stop. After 5 hours, the cellular surface markers were stained with anti-CD4-FITC, anti-IFNγ-PE, anti-IL4-PE, and anti-IL17A-APC antibodies. Each distribution of Th1, Th2, and Th17 cells was analyzed by fluorescence-activated cell sorting (FACS).

### 1.6. Confirmation of Cytokine Expression Level.

The splenocytes isolated from the spleen of each mouse model were cultured with anti-CD3/CD-28 antibodies for 72 hours. Then, the levels of inflammation-associated cytokine expressions were measured by using Th1/2/17 cytokine CBA kits. The Th1/2/17 cytokine were analyzed by using fluorescence-activated cell sorting (FACS) after the cytokine present in the culture medium reacted to beads bound with IL-2, IL-4, IL-6, IFN-γ, TNF-α, IL-10, and IL-17A antibodies.

### 1.7. Immunohistochemistry.

The mouse brain was fixed and dehydrated. Then, the brain was frozen and then cryosectioned at a thickness of 30 μm by using a frozen sectioning device. The sectioned brain slices were put into citrate buffer (pH 6.0) and treated at high-temperature. The antigen retrieval was carried out and followed by the treatment with 3% hydrogen peroxide for 10 min so as to remove intrinsic peroxidase activity. After blocking with 1.5% bovine serum albumin (BSA) in PBS for 1 hour, the result was treated with the anti-amyloid-beta antibodies (1:500 dilution) at 4° C. overnight. The result was treated with secondary antibodies and ABC reagents by using Vectastatin ABC kit. Then, DAB substrate was added to develop color.

### Example 2

Cognitive Ability Recovery in Animal Model Transplanted with Treg Cells.

Regulatory T cells (Treg) and effector T cells (Teff) were transplanted into 3xTg-AD mice, an Alzheimer's disease animal model. Then, Morris water maze experiment was conducted to test cognitive abilities thereof. In the Morris water maze experiment, the latency time when it took for each mouse model to find the hidden platform for 4 days of the training session; the retention time when the mouse had stayed in the platform; the time in quadrant when the mouse had stayed in the quadrant where the platform was located; and the number of crossing that the mouse had passed the location where the platform was installed; were measured.

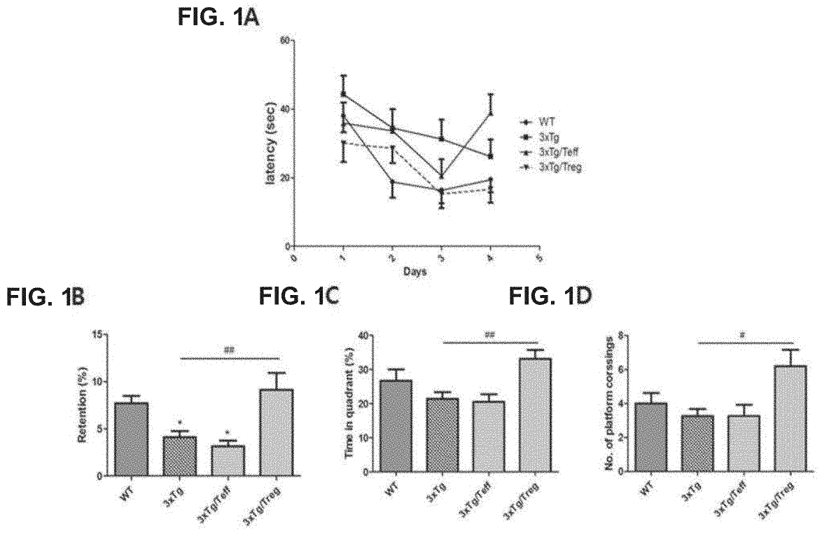

The results revealed that the cognitive ability of the mouse transplanted with Treg cells was improved compared with that of control group (WT, wild-type) (See FIG. 1).

### Example 3

Increase of Treg Cells and Regulation of Cytokines Expression in the Spleen of Animal Model Transplanted with Treg Cells.

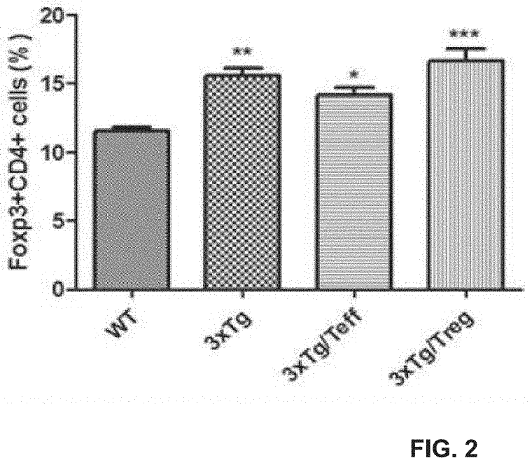

The distribution of Treg cells was examined in the spleen of each transplanted animal model so as to investigate whether immune cells were increased in the spleen and were directly related to immune responses in the case of transplantation of Treg cells and Teff cells. The results demonstrated that Treg cells (Foxp3+CD4+) were significantly increased in the spleen of the mouse transplanted with Treg cells (See FIG. 2).

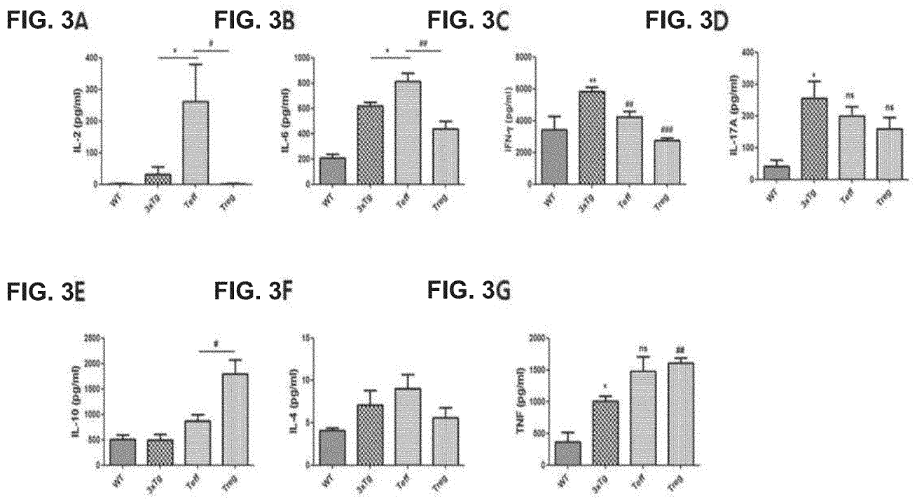

The expression levels of IL-2, IL-6, IFN-γ, IL-17A, IL-10, IL-4, and TNF-α were examined so as to investigate the expression level of inflammation-associated cytokine when Treg and Teff cells were transplanted. The results showed that Teff cell transplantation resulted in an increase in IL-2, IL-6, and TNF-α expressions compared to that of the 3xTg-AD mouse. However, Treg cells transplantation elicited a decrease in IL-6, IFN-γ, and IL-17A expression. In particular, IL-10, a major cytokine of Treg cells, was found to be significantly increased in the mouse transplanted with Treg cells (See FIG. 3).

### Example 4

Reduction of Amyloid-beta Plaques and Microglia in Animal Model Transplanted with Treg Cells.

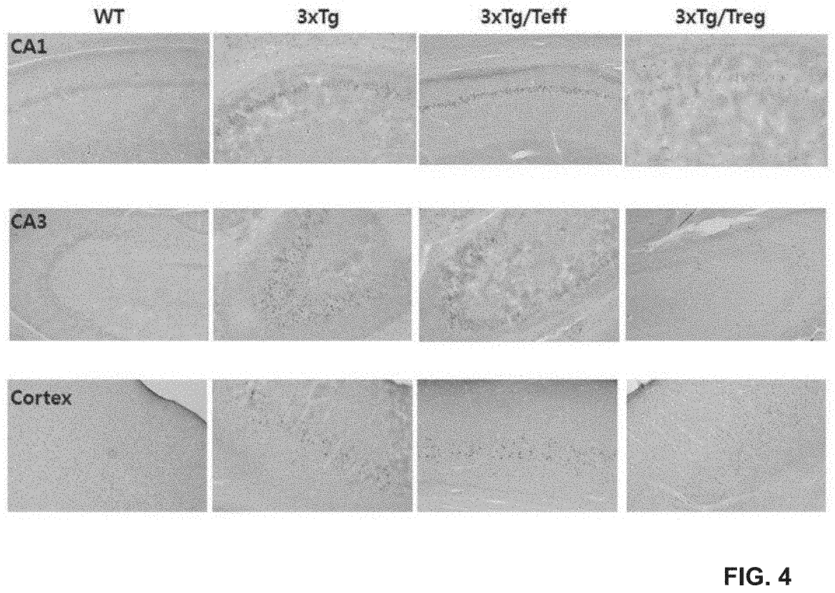

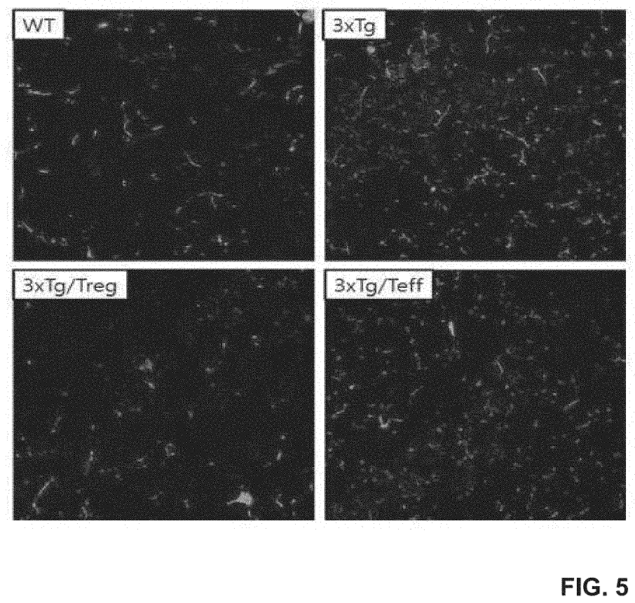

Amyloid-beta plaques were observed in the brain hippocampal region of 3xTg-AD mice transplanted with Treg cells and Teff cells. Compared with WT, the level of amyloid-beta plaques in the hippocampus of the mice transplanted with Teff cells was similar to that of the 3xTg-AD mice. However, amyloid-beta plaques and microglia were significantly reduced in the hippocampus of the mice transplanted with Treg cells (See FIGS. 4 and 5).

### Example 5

Cognitive Ability Recovery in Animal Model Transplanted with Amyloid-Beta Specific Treg Cells.

Amyloid-beta specific Treg cells were transplanted into 3xTg-AD mice, an Alzheimer's disease animal model. Then, Morris water maze experiments were conducted to test their cognitive abilities. In the Morris water maze experiment, the latency time when it took for each mouse model to find the hidden platform for 4 days of the training session; the retention time when the mouse had stayed in the platform; the time in quadrant when the mouse had stayed in the quadrant where the platform was located; and the number of crossing that the mouse had passed the location where the platform was installed; were measured.

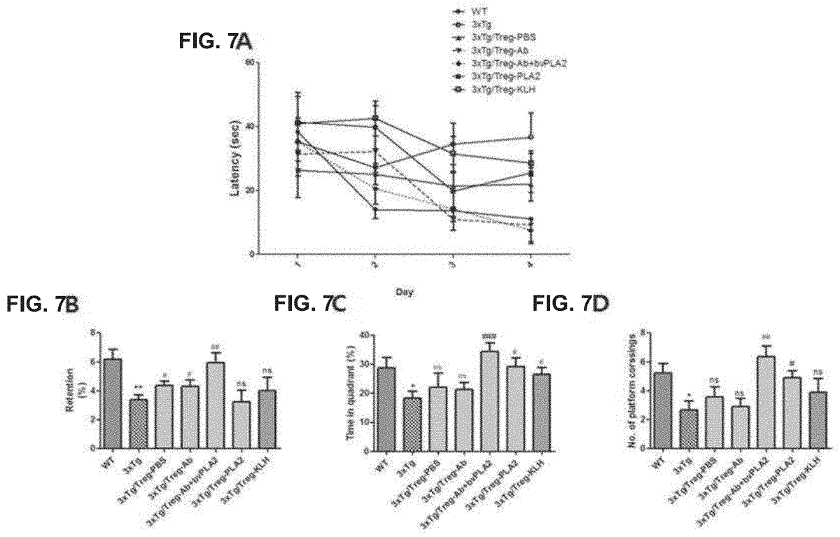

The results demonstrated that the level of cognitive ability was improved to a level similar to that of the control group in the mouse transplanted with amyloid-beta specific Treg cells and administered with bee venom phospholipase A2 (bvPLA2) at the same time (WT, wild-type) (See FIG. 7).

### Example 6

Increase of Helper T Cells and Regulation of Cytokines Expression in the Spleen of Animal Model Transplanted with Amyloid-Beta Specific Treg Cells.

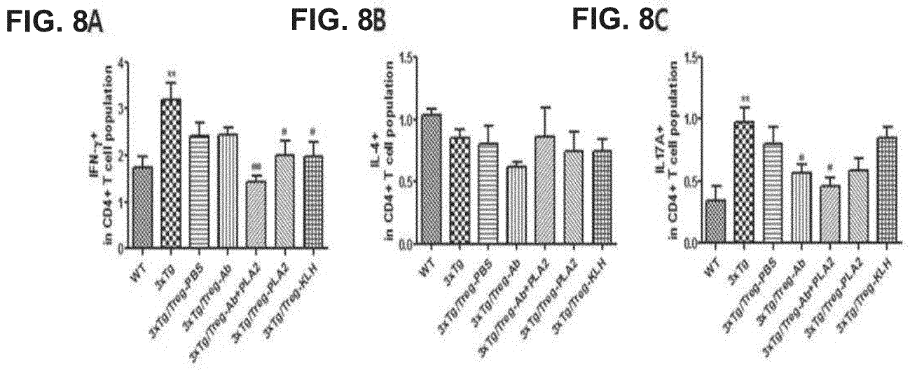

The distribution of helper T cells was examined in the case of administration of bee venom phospholipase A2 (bvPLA2) to 3xTg-AD mouse transplanted with amyloid-beta specific Treg cells. The results showed that when the amyloid-beta specific Treg cells were transplanted and the bee venom phospholipase A2 (bvPLA2) was administered, IFN-γ and IL-17A were decreased and IL-4 was increased in the CD4+ T cell group similarly to the control group (WT, wild-type) (See FIG. 8).

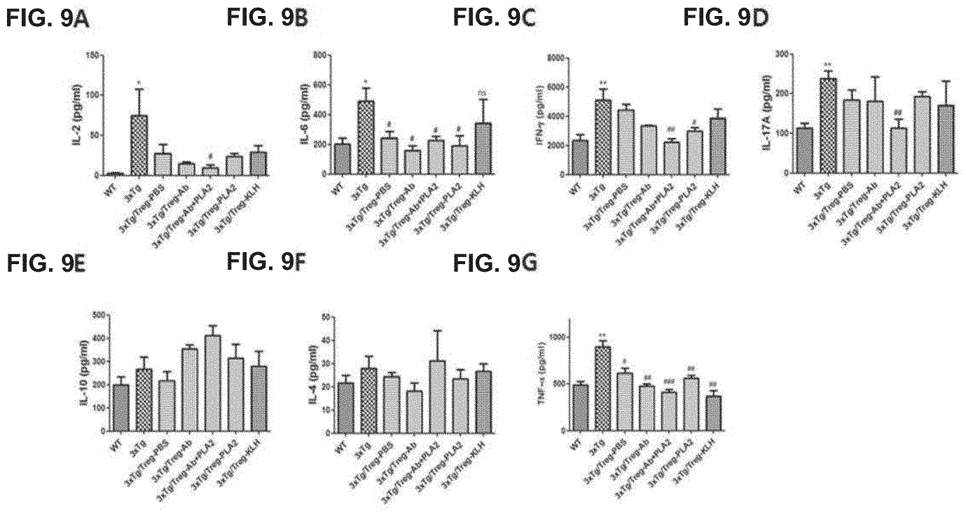

The expression levels of IL-2, IL-6, IFN-γ, IL-17A, IL-10, IL-4, and TNF-α were examined so as to investigate the expression level of inflammation-associated cytokine when bee venom phospholipase A2 (bvPLA2) was administered to the 3xTg-AD mouse transplanted with amyloid-beta specific Treg cells. The results revealed that the IL-2, IL-6, IFN-γ, IL-17A, and TNF-α expressions were decreased to a level similar to that of the control group (WT, wild-type), but the IL-10 and IL-4 expressions were increased (See FIG. 9).

### Example 7

Reduction of Amyloid-Beta Plaques in Animal Model Transplanted with Amyloid-Beta Specific Treg Cells.

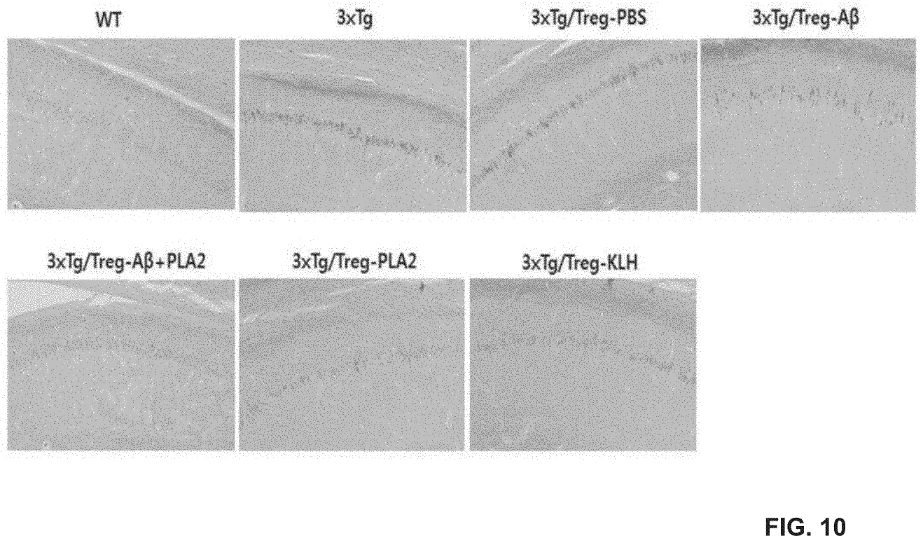

Amyloid-beta plaques were observed in the brain hippocampal region when the bee venom phospholipase A2 (bvPLA2) was administered to 3xTg-AD mouse transplanted with amyloid-beta specific Treg cells. The results showed that the amyloid-beta plaques were significantly reduced to a level similar to that of the control group (WT, wild-type) in the mouse transplanted with amyloid-beta specific Treg cells and administered with bee venom phospholipase A2 (bvPLA2) at the same time (See FIG. 10).

From the foregoing, it will be appreciated that various embodiments of the present invention have been described herein for purposes of illustration, and that various modifications may be made without departing from the scope and spirit of the present invention. Accordingly, the various embodiments disclosed herein are not intended to be limiting, with the true scope and spirit being indicated by the following claims.

What is claimed is:

1. A cell therapy method for treating neurodegenerative diseases, comprising administering a therapeutically effective amount of a regulatory T cell to an individual in need thereof.

2. The method of claim 1, wherein the therapeutically effective amount of the regulatory T cell is 1×10⁴ cells/kg to 1×10⁸ cells/kg.

3. The method of claim 1, wherein the regulatory T cell is administered intravenously, intraperitoneally, intramuscularly, subcutaneously or intradermally.

4. The method of claim 1, wherein the neurodegenerative diseases are selected from the group consisting of Alzheimer's disease, Huntington's disease, and Parkinson's disease.

5. The method of claim 1, wherein the regulatory T cell is an amyloid-beta specific regulatory T cell induced by administering amyloid-beta peptide and bee venom phospholipase A2 (bvPLA2).

6. The method of claim 5, wherein the amyloid-beta peptide consists of an amino acid sequence represented by SEQ ID NO: 1.

7. The method of claim 5, wherein the bee venom phospholipase A2 (bvPLA2) consists of an amino acid sequence represented by SEQ ID NO: 2.

8. The method of claim 5, wherein the therapeutically effective amount of the regulatory T cell is 1×10⁴ cells/kg to 1×10⁸ cells/kg.

9. The method of claim 5, wherein the regulatory T cell is administered intravenously, intraperitoneally, intramuscularly, subcutaneously or intradermally.

10. The method of claim 5, wherein the neurodegenerative diseases are selected from the group consisting of Alzheimer's disease, Huntington's disease, and Parkinson's disease.
